SEQUENCE LISTING (4 sequences)

SEQ ID NO: 1 - DNA, 20 bp, Artificial Sequence
  Synthetic DNA
atcaataagcggaggaaaag

SEQ ID NO: 2 - DNA, 20 bp, Artificial Sequence
  Synthetic DNA
actccttggtccgtgtttca

SEQ ID NO: 3 - DNA, 20 bp, Artificial Sequence
  Synthetic DNA
caacggatctcttggttctc

SEQ ID NO: 4 - DNA, 18 bp, Artificial Sequence
  Synthetic DNA
cccgcctgatttcagatc

(19) United States
(12) Patent Application Publication
(10) Pub. No.: US 2021/0235712 A1
(43) Pub. Date: Aug. 5, 2021

(54) FISH MEAT OXIDATION SUPPRESSION METHOD, PRESERVATION METHOD, TRANSPORTATION METHOD, DISCOLORATION SUPPRESSION METHOD AND FISHY SMELL SUPPRESSION METHOD, AND FISH MEAT

(71) Applicant: Meiji University, Tokyo (JP)

(72) Inventors: Shuichiro Murakami, Kawasaki-shi (JP); Mikio Atobe, Tokyo (JP)

(21) Appl. No.: 17/262,918

(22) PCT Filed: May 23, 2019
(86) PCT No.: PCT/JP2019/020421

§ 371 Date: Jan. 25, 2021

(30) Foreign Application Priority Data
Oct. 31, 2018 (JP) 2018-205243

(51) Int. Cl. A23B 4/22; A23B 4/06

(52) U.S. Cl. CPC A23B 4/22; A23B 4/06

(57) ABSTRACT

A fish meat oxidation suppression method, preservation method, transportation method, discoloration suppression method and fishy smell suppression method involve refrigerating the fish meat at a temperature of at least 0° C. but not more than 6° C. in a state where the fish meat is in contact with a substrate, wherein the substrate is provided with, at least in the portion that contacts the fish meat, fungi belonging to the genus Helicostylum, the genus Thamnidium, or the genus Mucor. The fish meat has a surface covered with the substrate, wherein the substrate is provided with, at least in the portion that contacts the fish meat, fungi belonging to the genus Helicostylum, the genus Thamnidium, or the genus Mucor.

### TECHNICAL FIELD

The present invention relates to a fish meat oxidation suppression method, preservation method, transportation method, discoloration suppression method and fishy smell suppression method, and fish meat. Priority is claimed on Japanese Patent Application No. 2018-205243, filed Oct. 31, 2018, the content of which is incorporated herein by reference.

### BACKGROUND ART

Fish are known to spoil from the internal organs and gills, and are typically subjected to preliminary processing to remove the internal organs and gills, and if necessary processed into a filleted state, before undergoing refrigerated storage at about 4° C. However, although varying depending on the type of fish, the preservation period is short, and is typically about one to three days. Conventionally, in order to enable long-term preservation of fish, methods such as salting or oil immersion, storage in ice water, or freezing methods have been used.

On the other hand, the inventors of the present invention have previously developed a method for producing aged meat that uses a cloth to which specific microbes have been adhered (for example, see Patent Document 1).

Among conventional fish preservation methods, with methods that employ salting or oil immersion, although long-term preservation of the fish is possible, because the product contains a large amount of salt or oil, food preparation methods are limited. Further, appreciating the inherent texture and flavor of the raw fish becomes difficult.

Ice water storage methods require a mass of ice water at least as large as the mass of the fish, resulting in increased transportation costs. Further, the preservation period is quite short, and similar to normal refrigerated storage.

In freezing methods, although long-term preservation of the fish is possible, damage caused by freezing and subsequent thawing, and dripping (excess moisture that escapes from the fish) during thawing mean that fishy odors tend to occur readily, which can cause detract from the taste of the fish.

Furthermore, although Patent Document 1 investigated a method for producing aged meats that included fish meat, a method for preserving and transporting fish while preventing spoiling of the fish is not known.

### PRIOR ART LITERATURE

### Patent Documents

Patent Document 1: Japanese Unexamined Patent Application, First Publication No. 2017-147950

Patent Document 2: Japanese Patent (Granted) Publication No. 3963306

### SUMMARY OF INVENTION

### Problems to be Solved by the Invention

The present invention has been developed in light of the above circumstances, and provides a novel fish meat oxidation suppression method, preservation method, transportation method, discoloration suppression method and fishy smell suppression method, and fish meat.

### Means for Solving the Problems

As a result of intensive research aimed at achieving the objects described above, the inventors of the present invention discovered that by refrigerating fish meat wrapped in a substrate to which specific microorganisms have been adhered, the fish meat could be preserved and transported while preventing putrefaction, oxidation, discoloration and the occurrence of odors such as fish odors, thus enabling them to complete the present invention.

In other words, the present invention includes the following aspects.

A fish meat oxidation suppression method according to a first aspect of the present invention is a fish meat oxidation suppression method that involves refrigerating the fish meat at a temperature of at least 0° C. but not more than 6° C. in a state where the fish meat is in contact with a substrate, wherein the substrate is provided with, at least in the portion that contacts the fish meat, fungi belonging to the genus Helicostylum, the genus Thamnidium, or the genus Mucor.

A fish meat preservation method according to a second aspect of the present invention is a fish meat preservation method that involves refrigerating the fish meat at a temperature of at least 0° C. but not more than 6° C. in a state where the fish meat is in contact with a substrate, wherein the substrate is provided with, at least in the portion that contacts the fish meat, fungi belonging to the genus Helicostylum, the genus Thamnidium, or the genus Mucor.

The material for the substrate may be rayon or cotton.

A fish meat transportation method according to a third aspect of the present invention is a fish meat transportation method that involves refrigerating the fish meat at a temperature of at least 0° C. but not more than 6° C. in a state where the fish meat is in contact with a substrate, wherein the substrate is provided with, at least in the portion that contacts the fish meat, fungi belonging to the genus Helicostylum, the genus Thamnidium, or the genus Mucor.

A fish meat discoloration suppression method according to a fourth aspect of the present invention is a fish meat discoloration suppression method that involves refrigerating the fish meat at a temperature of at least 0° C. but not more than 6° C. in a state where the fish meat is in contact with a substrate, wherein the substrate is provided with, at least in the portion that contacts the fish meat, fungi belonging to the genus Helicostylum, the genus Thamnidium, or the genus Mucor.

A fish meat fishy smell suppression method according to a fifth aspect of the present invention is a method that involves refrigerating the fish meat at a temperature of at least 0° C. but not more than 6° C. in a state where the fish meat is in contact with a substrate, wherein the substrate is provided with, at least in the portion that contacts the fish meat, fungi belonging to the genus Helicostylum, the genus Thamnidium, or the genus Mucor.

Fish meat according to a sixth aspect of the present invention is fish meat that has the surface covered with a substrate, wherein the substrate is provided with, at least in the portion that contacts the fish meat, fungi belonging to the genus Helicostylum, the genus Thamnidium, or the genus Mucor.

### Effects of the Invention

By using the fish meat oxidation suppression method of the aspect described above, oxidation of various components (and particularly fats) contained in the fish meat can be effectively suppressed. By using the fish meat preservation method of the aspect described above, the fish meat can be stored under refrigeration while preventing spoiling. By using the fish meat transportation method of the aspect described above, the fish meat can be transported while preventing spoiling. By using the fish meat discoloration suppression method of the aspect described above, discoloration of the fish meat can be effectively suppressed. By using the fish meat fishy smell suppression method of the aspect described above, the occurrence of fish odors can be effectively suppressed. Further, by using the fish meat of the aspect described above, fish meat that exhibits little spoiling and with good suppression of discoloration, fish odors and oxidation can be provided.

### BRIEF DESCRIPTION OF THE DRAWINGS

FIG. 1 is an image showing a sample to which has been affixed a substrate onto which spores have been adhered, on the 14th day of storage in Example 3.

FIG. 2 is an image showing a sample to which has been affixed a substrate onto which spores have been adhered, on the 21st day of storage in Example 3.

FIG. 3 is a graph illustrating the results of a TBARS (2-thiobarbituric acid reactive substance) assay in Example 3.

### EMBODIMENTS FOR CARRYING OUT THE INVENTION

### «Fish Meat Preservation Method»

The fish meat preservation method of the present embodiment (hereafter sometimes abbreviated as simply “the preservation method of the present embodiment”) is a preservation method that involves refrigerating the fish meat at a temperature of at least 0° C. but not more than 6° C. in a state where the fish meat is in contact with a substrate. Further, the substrate is provided with, at least in the portion that contacts the fish meat, fungi belonging to the genus Helicostylum, the genus Thamnidium, or the genus Mucor (hereafter sometimes abbreviated as simply “the fungi”).

In the preservation method of the present embodiment, by bringing the substrate described above into contact with the fish meat and then performing refrigerated storage, the fish meat can be stored while preventing spoiling. Further, with conventional refrigerated storage, the preservation period is typically at least 1 but not more than 3 days. Furthermore, generally, if fish meat is stored under refrigeration for 10 days or longer, then oxidation odors become severe, and a state not suitable for food is reached. In contrast, by using the preservation method of the present embodiment, the inherent texture and flavor of the raw fish can be enjoyed beyond the conventional preservation period. In the case of even longer storage, the texture and flavor of fish meat (and particularly sablefish) that has been aged by the fungi can still be enjoyed.

In the preservation method of the present embodiment, the fish meat is first wrapped in the substrate. The fish meat may also be subjected to a pretreatment such as bringing the fish meat into contact with alcohol or a low-concentration sodium chloride aqueous solution of about 0.1% to 1.0% by weight. Examples of the method used for bringing the fish meat into contact with the alcohol or sodium chloride aqueous solution include dipping methods and spraying methods.

Subsequently, the fish meat in contact with the substrate is stored under refrigeration. Examples of the method used for bringing the substrate into contact with the fish meat include methods in which the substrate is wrapped around the fish meat to cover the surface of the fish meat, and methods in which a plurality of substrate sheets are laid over the surface of the fish meat in contact with the fish meat.

In terms of the storage conditions, the temperature is typically at least 0° C. but not more than 6° C., preferably at least 0° C. but not more than 5° C., and more preferably at least 0° C. but not more than 4° C. By ensuring that the storage temperature falls within this range, the fungi can be more easily grown, whereas the growth of other unwanted microorganisms such as putrefying bacteria can be suppressed.

In terms of humidity, there are no problems with the degree of humidity in a typical refrigerator, and for example, a humidity of at least 40% RH but not more than 90% RH is preferred.

### <Substrate>

The substrate is preferably composed of material and form on which the fungi can grow and through which oxygen can permeate (has oxygen permeability). Further, the substrate must not exude harmful components or the like even when in contact with fish meat. In terms of ease of handling, the substrate is preferably in sheet form. When the substrate is in sheet form, the entire surface of the fish meat can be covered.

Examples of the material for the substrate include cotton, silk, linen, rayon, acetate, cupra, nylon, polyurethane, polyester and acrylic. Among these, in terms of facilitating adhesion of the fungi and being inexpensive to obtain, rayon or cotton is preferred.

### [Fungi]

The fungi adhered to the substrate is fungi belonging to the genus Helicostylum, the genus Thamnidium, or the genus Mucor. These fungi are filamentous microorganisms. In general, the term filamentous microorganisms is a generic term for microorganisms composed of tubular cells known as mycelia. Further, the fungi must be adhered to at least the portion of the substrate that contacts the fish meat, but may be adhered to the entire substrate. Further, a single type of fungi belonging to one of the genera described above may be used alone, or a combination of two or more types may be used.

Examples of fungi belonging to the genus Helicostylum include Helicostylum pulchrum and Helicostylum elegans. One of these fungal strains may be used alone, or a combination of two or more strains may be used.

Examples of fungi belonging to the genus Thamnidium include Thamnidium elegans and the like. One of these fungal strains may be used alone, or a combination of two or more strains may be used.

Examples of fungi belonging to the genus Mucor include Mucor aligarensis and Mucor flavus. One of these fungal strains may be used alone, or a combination of two or more strains may be used.

### [Other Components]

Besides the fungi, the substrate may also contain carbohydrates and minerals and the like. Among the various possibilities, the substrate preferably contains carbohydrates. By including carbohydrates, the carbohydrates can be used as nutrients for growth of the fungi, so that even on regions of the fish meat having high fat contents where growth of the fungi is difficult, the fungi can still grow efficiently, enabling the fish meat to be preserved in a stable manner.

Any carbohydrate that can be assimilated by the cells of the fungi may be used, and examples include monosaccharides, disaccharides, oligosaccharides, polysaccharides, and sugar alcohols and the like. Examples of the monosaccharides include glucose, fructose, mannose, galactose, ribose, sorbose and ribulose. Examples of the disaccharides include lactose, maltose and sucrose. Examples of the oligosaccharides include raffinose, maltotriose, fructooligosaccharides, galactooligosaccharides and mannan oligosaccharides. Examples of the polysaccharides include starch, cellulose and glycogen. Examples of the sugar alcohols include glycerol, erythritol, lactitol, maltitol, mannitol, sorbitol and xylitol.

Furthermore, examples of the minerals include magnesium ions, potassium ions and sodium ions.

### <Fish Meat>

In this description, the term “fish meat” means the edible portions of seafood. Examples of the seafood include fish, shellfish, other aquatic animals and marine mammals.

Although not limited to the following, examples of the fish include catadromous migratory fish, anadromous migratory fish, freshwater amphidromous fish, saltwater amphidromous fish, and saltwater fish. Examples of catadromous migratory fish include eels and the like. Examples of anadromous migratory fish include salmon, trout, shishamo smelt, and Arctic lamprey. Examples of freshwater amphidromous fish include ayu and gobies. Examples of saltwater amphidromous fish include flathead mullet and Japanese lates. Examples of saltwater fish include fish belonging to the family Clupeidae, the family Scombridae, the family Carangidae, the family Coryphaenidae, the family Gadidae, the family Pleuronectidae, the family Paralichthyidae, the family Serranidae, the family Sparidae, the family Sciaenidae, the family Anoplopomatidae, the order Lamniformes and the order Squaliformes. Examples of fish belonging to the family Clupeidae include herrings and sardines. Examples of fish belonging to the family Scombridae include bonito, tuna, mackerel and Spanish mackerel. Examples of fish belonging to the family Carangidae include horse mackerel and yellowtail. Examples of fish belonging to the family Coryphaenidae include mahi-mahi. Examples of fish belonging to the family Gadidae include Pacific cod, Alaska pollack and saffron cod. Examples of fish belonging to the family Pleuronectidae include dab. Examples of fish belonging to the family Paralichthyidae include flounder. Examples of fish belonging to the family Serranidae include sea bass. Examples of fish belonging to the family Sparidae include red sea bream. Examples of fish belonging to the family Sciaenidae include croaker.

Although not limited to the following, examples of the shellfish include shellfish belonging to the family Corbiculidae, the family Viviparidae, the family Ostreidae, the family Gryphaeidae, the family Pectinidae, the family Arcidae, the family Veneridae, the family Mactridae, the family Haliotidae, and the family Turbinidae. Examples of shellfish belonging to the family Arcidae include the ark shell and ark clam (mogai). Examples of shellfish belonging to the family Veneridae include the Asian hard clam and Japanese littleneck. Examples of shellfish belonging to the family Haliotidae include abalone.

Although not limited to the following, examples of the other aquatic animals include molluscs, crustaceans, echinoderms, and aquatic animals belonging to the order Testudinata. Examples of the molluscs include squid and octopus. Examples of the crustaceans include shrimps, crabs and crayfish. Examples of the echinoderms include sea urchins, sea cucumbers and starfish.

Although not limited to the following, examples of the marine mammals include sea lions, dolphins and whales.

Among the various possibilities, in terms of the supply of fish meat, the preservation method of the present embodiment is preferably used with large fish species which yield edible portions of at least 1.0 kg following trimming, such as tuna, marlin, bonito, yellowtail, cod and sablefish. By using the preservation method of the present embodiment, these large fish species can be stored under refrigeration in a raw (unheated) state with good maintenance of quality.

Further, the seafood may be natural or farmed.

Furthermore, the processed form of the fish meat may include rounds, semi-dressed, dressed, center cuts, fillets, loins, belly flaps, slices, sashimi (but excluding assorted mixtures), shucked shellfish, and thawed portions obtained from the above forms in a frozen state. Among the various possibilities, in terms of ensuring that the effects disclosed in this description manifest prominently upon bringing the fungi adhered to the substrate into direct contact with a portion of flesh (and particularly the flesh around the backbone), forms including semi-dressed, dressed, center cuts, fillets, loins, belly flaps, slices, sashimi (but excluding assorted mixtures), shucked shellfish, and thawed portions obtained from the frozen state of these forms are preferred.

Rounds, semi-dressed, dressed, center cuts, fillets, loins, and belly flaps mainly refer to processed forms of fish. A “round” indicates a whole fish in an unprocessed state. The term “semi-dressed” indicates a fish from which the internal organs have been removed. The term “dressed” indicates a fish from which the head and the internal organs have been removed. A “center cut” indicates a dressed fish that has been cut open along the backbone. A “fillet” indicates a portion of dressed fish from which the tail, fins and backbone have been removed. A “loin” is fish meat composed of the dorsal portion of a dressed fish. A “belly flap” is fish meat composed of the belly portion of a dressed fish.

Furthermore, a “slice” describes seafood that has been cut to a standard grammage. Moreover, “sashimi” indicates raw seafood that has been thinly sliced.

Further, in the preservation method of the present embodiment, for fish which are known to have a high histidine content, such as tuna, marlin, bonito, mackerel, sardines, saury, yellowtail and horse mackerel, the production of histamines during the preservation period (from 10 days to preferably 28 days) can be suppressed in the fish meat following trimming of several mm to about 1 cm of meat from the surface to which the fungi were adhered. It is thought this is because growth of the fungi occurs predominantly, with growth of histamine-producing bacteria being suppressed.

Fish meat that has been preserved using the preservation method of the present embodiment can be eaten by trimming several mm to about 1 cm of meat from the surface to which the fungi were adhered.

### <Method for Producing Substrate>

Examples of the method used for producing the substrate include the method disclosed in Patent Document 1, and a specific method is as described below.

First, a suspension of fungal spores is prepared (the suspension preparation step), and that suspension is then brought into contact with a substrate of appropriate size to adhere the fungi to the substrate (the adhesion step). The temperature is preferably room temperature (for example, at least 20° C. but not more than 30° C.). The contact time may be adjusted appropriately depending on the size of the substrate. Further, the suspension may also contain components (such as carbohydrates and minerals) that can function as nutrients for the fungi. Furthermore, a method in which the suspension is sprayed onto the substrate using a sprayer or the like may be used to adhere the fungi, but in order to ensure uniform adhesion of the fungi to the substrate, a method in which the substrate is dipped in the suspension is preferred. Subsequently, by drying the substrate to which the fungi have been adhered using a dryer (the drying step), the substrate with adhered fungi can be obtained. The drying temperature and drying time may be adjusted appropriately in accordance with the size of the substrate.

### «Fish Meat Transportation Method»

The fish meat transportation method of the present embodiment (hereafter sometimes abbreviated as simply “the transportation method of the present embodiment”) is a transportation method that includes refrigerating the fish meat at a temperature of at least 0° C. but not more than 6° C. in a state where the fish meat is in contact with a substrate. Further, the substrate is provided with, at least in the portion that contacts the fish meat, fungi belonging to the genus Helicostylum, the genus Thamnidium, or the genus Mucor.

By using the transportation method of the present embodiment, fish meat can be transported while preventing spoiling.

Further, conventionally, when transporting fish meat, the fish meat was required to be held in an amount of ice water of similar mass to the fish meat. In contrast, in the transportation method of the present embodiment, by wrapping the fish meat with the substrate, there is no need to use the ice water mentioned above, meaning the distribution costs associated with the ice water can be eliminated.

The transportation method of the present embodiment can be used favorably with large fish species such as tuna. By using the transportation method of the present embodiment, the quality of large fish species can be maintained in a raw (unheated) state, and long-distance transport such as international transport can be conducted with reduced transportation costs.

The fish meat preservation conditions and the substrate and fungi used in the transportation method of the present embodiment are the same as described above in relation to the “fish meat preservation method”.

### «Fish Meat Oxidation Suppression Method»

The fish meat oxidation suppression method of the present embodiment (hereafter sometimes abbreviated as simply “the oxidation suppression method of the present embodiment”) is an oxidation suppression method that involves refrigerating the fish meat at a temperature of at least 0° C. but not more than 6° C. in a state where the fish meat is in contact with a substrate. Further, the substrate is provided with, at least in the portion that contacts the fish meat, fungi belonging to the genus Helicostylum, the genus Thamnidium, or the genus Mucor.

By using the oxidation suppression method of the present embodiment, oxidation of various components (and particularly fats) contained in the fish meat can be effectively suppressed.

Conventionally, as the storage period for fish meat lengthens, various components contained in the fish meat undergo oxidation, producing a state not suitable for food. In particular, oxidation of fats contained in fish meat causes a marked deterioration in the taste. In contrast, in the oxidation suppression method of the present embodiment, by effectively suppressing the oxidation of various components such as the fats mentioned above, the inherent texture and flavor of the raw fish can be maintained beyond the conventional storage period. This is also illustrated in the examples described below, in which the inventors of the present invention were able to clearly demonstrate that by using the oxidation suppression method of the present embodiment, the amount of aldehyde-like substances (such as malondialdehyde) produced by oxidation of the fats was markedly reduced, with the fish meat that used the oxidation suppression method of the present embodiment having a low degree of oxidation of fats.

The detailed mechanism by which oxidation of the fish meat is suppressed remains unclear, but it is thought that the fungi metabolize or decompose various substances (and particularly fats) contained in the fish meat into other materials, thereby suppressing the production of oxidized products of the various substances (and particularly aldehyde-like substances produced by oxidation of fats).

The fish meat preservation conditions and the substrate and fungi used in the oxidation suppression method of the present embodiment are the same as described above in relation to the “fish meat preservation method”.

### «Fish Meat Discoloration Suppression Method»

The fish meat discoloration suppression method of the present embodiment (hereafter sometimes abbreviated as simply “the discoloration suppression method of the present embodiment”) is a discoloration suppression method that involves refrigerating the fish meat at a temperature of at least 0° C. but not more than 6° C. in a state where the fish meat is in contact with a substrate. Further, the substrate is provided with, at least in the portion that contacts the fish meat, fungi belonging to the genus Helicostylum, the genus Thamnidium, or the genus Mucor.

By using the discoloration suppression method of the present embodiment, discoloration of the fish meat can be effectively suppressed.

The color of fish meat generally depends on the type of pigments that exist in the skin and muscles of the fish. In this description, examples of “fish meat discoloration” include fading of the color of carotenoid-based pigments (such as astaxanthin, tunaxanthin and lutein) that exist in the skin and muscles of seafood, black discoloration caused by the accumulation of melanin polymers produced by the oxidation of tyrosine in crustaceans, browning (and in particular, marked discoloration in red-fleshed fish) caused by oxidation of myoglobin, hemoglobin and their derivatives which exist in the muscles of seafood, oil staining caused by the oxidation of seafood that contains large amounts of subcutaneous fat, and yellowish brown discoloration (and in particular, marked discoloration in white-fleshed fish) caused by the accumulation of melanoidin produced by a Maillard reaction between reducing sugars and amino compounds contained in seafood. By using the discoloration suppression method of the present embodiment, these types of fish meat discoloration can be effectively suppressed.

The discoloration suppression method of the present embodiment is particularly effective for red-fleshed fish. Further, even in white-fleshed fish, discoloration of the dark flesh (the muscles that run along the central portion beneath the skin on both sides of the fish) can be effectively suppressed. Examples of red-fleshed fish include bonito, tuna, yellowtail, horse mackerel, sardines, saury and mackerel. Examples of white-fleshed fish include sea bream, cod, flounder and halibut.

The fish meat preservation conditions and the substrate and fungi used in the discoloration suppression method of the present embodiment are the same as described above in relation to the “fish meat preservation method”.

Further, although the detailed mechanism is unclear, the inventors of the present invention also discovered that for fish meat that had undergone long-term refrigerated storage of 28 days or the like using the discoloration suppression method of the present embodiment, even when the substrate was removed, several mm to about 1 cm of fish meat was trimmed from the surface, and the trimmed fish meat was then refrigerated for a further period of about 5 days at a temperature of at least 0° C. but not more than 6° C., the state of suppressed discoloration of the fish meat was maintained.

### «Fish Meat Fishy Smell Suppression Method»

The fish meat fishy smell suppression method of the present embodiment (hereafter sometimes abbreviated as simply “the fishy smell suppression method of the present embodiment”) is a fishy smell suppression method that involves refrigerating the fish meat at a temperature of at least 0° C. but not more than 6° C. in a state where the fish meat is in contact with a substrate. Further, the substrate is provided with, at least in the portion that contacts the fish meat, fungi belonging to the genus Helicostylum, the genus Thamnidium, or the genus Mucor.

By using the fishy smell suppression method of the present embodiment, the occurrence of fish odors can be effectively suppressed.

In this description, examples of fish odors (fishy smell) include raw fish odors (main causative agents: trimethylamine, piperidine, acetic acid, butyric acid, and valeric acid and the like), oxidized odors (main causative agents: peroxidized fats and the like), and cultivation odors. Here, the term “cultivation odors” refers to odors peculiar to seafood that has been farmed (hereafter sometimes abbreviated as “farmed seafood”). It is known that one of the causes of cultivation odors is odor associated with the administered feed. For example, in fanned seafood that has eaten blue-green algae which produces diosmin (for example, see Patent Document 2) and 2-methylisoborneol (2-MIB), those substances have been clearly detected.

The detailed mechanism by which the occurrence of odors is suppressed remains unclear, but it is thought that the fungi metabolize or decompose substances contained in the fish meat into other materials, thereby suppressing the production of various substances that can cause fish odors.

Among the various possibilities, the fishy smell suppression method of the present embodiment is particularly effective in suppressing cultivation odors, and is therefore ideal for use with farmed seafood.

The fish meat preservation conditions and the substrate and fungi used in the fishy smell suppression method of the present embodiment are the same as described above in relation to the “fish meat preservation method”.

### EXAMPLES

The present invention is described below using a series of examples, but the present invention is not limited by the following examples.

### Example 1

### 1. Preparation of Fungi

(1) Screening of Microorganisms from Aged Meat

### (1-1) Preparation of Screening Medium

A 200-mL flask was charged with 3.9 g of a potato dextrose agar medium (manufactured by Nissui Pharmaceutical Co., Ltd.) and 100 mL of distilled water, the medium was dissolved by heating in a hot water bath with constant stirring, and was then autoclaved (high-pressure steam sterilization treatment: 121° C., 15 minutes). Subsequently, the autoclaved medium was subjected to UV irradiation for 15 minutes in a clean bench, and then dispensed into a sterilized plastic petri dish (diameter: 90 mm), thus producing a potato dextrose agar (PDA) plate for screening purpose. Further, a plate of the same composition containing 0.01% of Triton X-100 was produced in the same manner as the medium used for pure separation of isolates. Furthermore, a PDA medium dispensed into a test tube (PDA slant) was also prepared for isolation and storage the isolates.

### (1-2) Screening of Microorganisms

Subsequently, the preferentially grown microorganisms on aged meat (dressed meat) in an aging chamber were picked up using a dry heat-sterilized tweezer or a platinum loop and spread on a potato dextrose agar plate for screening. The plate was then cultured at 4° C. and 15° C. for 3 to 4 days, and the grown filamentous fungi like microorganisms were collected and applied to a plate used for pure separation. Subsequently, the fungi grown in a similar manner were collected in the PDA slant isolates. Further, a suspension obtained by suspending the spores in an autoclaved 20% (w/v) glycerol solution was stored at −80° C. as a glycerol stock.

### (2) Identification of Isolated Fungal Strain

### (2-1) Preparation of Medium for Identification of Fungal Strain

In order to prepare a medium for harvest of cells from filamentous microorganisms, a 200-mL flask was charged with 1 g of a yeast extract (manufactured by Difco Laboratories), 1 g of polypepton (manufactured by Nihon Pharmaceutical Co., Ltd.), 2 g of D-glucose and 80 mL of distilled water, and following complete dissolution under stirring, the volume was made up to 100 mL Subsequently, 100 mL of the medium was dispensed into test tubes with 5 mL in each tube, and following autoclave treatment, the medium was used as a PGY (Peptone, Glucose, Yeast extract) liquid medium for harvest of isolated fungal cells.

Further, in order to prepare a medium for E. coli transformants used in identification testing, a flask was charged with 0.5 g of a yeast extract (manufactured by Difco Laboratories), 1 g of polypepton (manufactured by Nihon Pharmaceutical Co., Ltd.), 1 g of sodium chloride and 80 mL of distilled water, and the mixture was stirred and dissolved. Subsequently, the pH was adjusted to 6.8 to 7.0 using a 1 N aqueous solution of sodium hydroxide. The volume was then made up to 100 mL, and 2 mL of the medium were dispensed into test tubes.

When preparing an agar medium, 1.5 g of agar was added to the medium after making up and dissolved using a hot water bath. After autoclaving, the agar medium was placed in a clean bench that had been subjected to UV irradiation for 15 minutes, and sodium ampicillin (50 μg/mL), isopropyl-β-D(−)-thiogalactopyranoside (1 mM) and 5-bromo-4-chloro-3-indolyl-β-D-galactopyranoside (0.04%) were added to the agar medium in sufficient amounts to achieve final concentrations shown inside the respective parentheses, and the resulting mixture was then dispensed into a sterilized plastic petri dish and used as an LB agar plate for isolation of transformants.

Prior to cultivation for plasmid recovery, sodium ampicillin was added to 2 mL of the LB liquid medium at the final concentration of 50 μg/mL, and the medium was then used for cultivation.

### (2-2) Identification of Fungal Strain

The isolated fungal strain was identified using the method described below, based on DNA sequencing of an internal transcribed spacer (ITS, described below) and 28S rRNA gene.

(2-2-a) Preparation of DNA from Fungal Cells

The fungal cells were cultivated in PGY liquid medium at 15° C. for 2 days, and harvested using a Buchner funnel fitted with a miracloth. Subsequently, according to a conventional method (reference document: Makiko Hamamoto, “Classification and Identification test Methods for Microbes—centered on Molecular Genetics and Biological Techniques”, 2. DNA Preparation, 2.2. Yeast and filamentous Bacteria, 2.2.3. Small-Scale Methods, pp. 26 to 27, published by Springer-Verlag Tokyo, Inc., 2001), DNA was extracted from the fungal cells. The concentration of the extracted DNA was confirmed by 1% agarose gel electrophoresis. HindIII-digested 2-DNA was used as a size marker.

(2-2-b) Amplification of 28S rRNA Gene by PCR

Subsequently, according to a conventional method (reference document: Sandhu, G. S., Kline, B. C., Stockman, L. and Roberts, G. D., “Molecular probes for diagnosis of fungal-infections”, Journal of Clinical Microbiology, 33, 2913 to 2919, 1995), a portion of the 28S rRNA gene was amplified by PCR using the DNA prepared above in (2-2-a) as a template. A P1 primer (SEQ ID NO: 1: 5′-ATCAATAAGCGGAGGAAAAG-3′) and a P4 primer (SEQ ID NO: 2: 5′-ACTCCTTGGTCCGTGTTTCA-3′), which represent specific sequences in the 28S rRNA gene of the fungus, were used as primers. Confirmation of the amplification was performed by 2% (w/v) agarose gel electrophoresis. A 100 bp DNA ladder (manufactured by Bioneer Inc.) was used as a size marker.

(2-2-c) Purification of PCR Products

The PCR products were purified using a FavorPrep GEL/PCR Purification Mini Kit (manufactured by FAVORGEN Biotech Corporation). Subsequently, an ethanol precipitation described below was conducted. To the PCR products obtained above in (2-2-b) were added 1/10-fold volume of 3 M acetate buffer (pH 5.2) and 2.5-fold volume of 99.5% EtOH, and after incubation at 25° C. for 15 minutes, the mixture was centrifuged at 15,400×g and 15 minutes for 25° C. The supernatant was then removed, 100 μL of 70% ethanol was added, and centrifuged at 15,400×g and 25° C. for 10 minutes. Subsequently, the supernatant was removed, and the precipitate was dried by vacuum for 10 minutes (hereafter, this series of operations is termed “ethanol precipitation”). Subsequently, the obtained precipitate was dissolved in 4 μL of a TE buffer (10 mM Tris-HCl (pH 8.0), 1 mM EDTA). Of the resulting solution, 1 μL was submitted to 2% (w/v) agarose gel electrophoresis, confirming the recovery of the purified PCR products.

(2-2-d) Ligation of Vector and Purified PCR Products

Using the purified PCR products and the pGEM T-Easy Vector Systems I (manufactured by Promega Corporation), overnight incubation was conducted at 12° C. to perform a ligation reaction.

(2-2-e) Transformation of E. coli and Recovery of Plasmid

ECOS (a registered trademark) Competent E. coli DH5a (manufactured by Nippon Gene Co., Ltd.) was transformed in accordance with the product manual using the ligation mixture prepared above in (2-2-d). The thus obtained transformant was then cultivated on LB agar plates at 37° C. for 20 hours. Subsequently, the grown colony was transferred into 2 mL of the LB liquid using a sterilized toothpick, and cultivated at 37° C. for 16 hours. Subsequently, using a conventional method (reference document: Sambrook, J. and Russell, D. W., Molecular cloning: a laboratory manual, 3rd ed., “Preparation of plasmid DNA by Alkaline Lysis with SDS: Minipreparation”, pp. 1.32 to 1.34, Cold Spring Harbor Laboratory Press, Cold Spring Harbor, N.Y., 2001), the plasmid DNA was recovered by the alkaline lysis method. The plasmid DNA was then dissolved in 40 μL of TE buffer containing 0.2 μL of an RNase A solution (10 mg/mL, manufactured by Sigma-Aldrich Corporation). Subsequently, an incubation was conducted at 37° C. for 5 minutes, and 1 μL of the resulting solution was submitted to 1% (w/v) agarose gel electrophoresis, confirming the collection of the plasmid DNA of the target size. HindIII-digested λ-DNA was used as a size marker.

(2-2-f) DNA Sequencing

Subsequently, the plasmid DNA recovered above in (2-2-e) was purified using a FavorPrep GEL/PCR Purification Mini Kit (manufactured by FAVORGEN Biotech Corporation). A 1 μL of the purified plasmid DNA solution was submitted to 1% (w/v) agarose gel electrophoresis, confirming the recovery of the plasmid DNA. Subsequently, 4 μL of the remaining plasmid DNA was used for sequencing. Using a BigDye (a registered trademark) Terminator v3.1 Cycles Sequencing Kit (manufactured by Applied Biosystems, Inc.), cycle sequencing was performed. First, the plasmid DNA was amplified by a PCR. The PCR conditions included 35 repetitions of a cycle that involved heating at 96° C. for one minute, followed by 10 seconds at 96° C., 5 seconds at 50° C. and 4 minutes at 60° C. Subsequently, the reaction mixture containing the PCR products was subjected to ethanol precipitation and then dissolved in 15 μL of Hidi formamide (manufactured by Applied Biosystems, Inc.), and the thus obtained samples were applied to a 3130 Genetic Analyzer (manufactured by Applied Biosystems, Inc.) to obtain the DNA sequence. The obtained data were analyzed using Chromas LITE version 2.01 (manufactured by Technelysium Pty. Ltd.) and GENETYX (a registered trademark) WIN version 3.1.0 (manufactured by Software Development Co., Ltd.). Sequences having identity with the determined sequence were searched at the European Molecular Biology Laboratory database (http://www.ebi.ac.uk/embl/) using the fasta program. The isolated fungal strain was identified on the basis of the degree of identity.

Based on the fungal morphotype and the analysis of the 28S rRNA gene sequence, it was found that fungal of a number of genera belonging to the family Mucoraceae of the order Mucorales exhibited high identities. Among the genera candidates having high identities, in order to identify the fungal strain designated JW-1 from among the isolated fungi, identification was conducted using the Internal Transcribed Spacer (ITS) region. The candidate strain was selected based on genera disclosed in a reference document (Walther et al., “DNA barcoding in Mucorales: an inventory of biodiversity”, Persoonia, 30, 2013: 11 to 47). Specifically, the identification was performed by DNA preparation from the fungal cells, PCR, purification of the PCR products, and DNA sequencing using the direct sequencing method. Primers specific to the amplification of the ITS region from fungal species exhibiting high identities (JW-ITS-F (SEQ ID NO: 3): 5′-CAACGGATCTCTTGGTTCTC-3′, JW-ITS-R (SEQ ID NO: 4): 5′-CCCGCCTGATTTCAGATC-3′) were designed and used. The results are shown in Table 1.

TABLE 1

| Candidate | Accession No. | Identity (%) |
|---|---|---|
| Helicostylum pulchrum CBS 259.68 | JN206052 | 100% |
| Helicostylum elegans CBS 169.57 | AB113013 | 99.5% |
| Thamnidium elegans CBS 341.55 | JN206060 | 94.5% |
| Mucor aligarensis CBS 993.70 | JN206051 | 93.6% |
| Mucor flavus CBS 234.35 | JN206056 | 91.4% |
| Pirella circinans CBS 962.68 | JN206102 | 80.3% |

From Table 1, it was evident that strain JW-1 exhibited an identity of at least 99.5% with both species of the genus Helicostylum (identity with Helicostylum pulchrum CBS 259.68 (Accession No. JN206052): 100%), and had an identity of no higher than 94.5% with species of the genus Thamnidium and the genus Mucor. Based on these results, strain JW-1 was identified as fungus belonging to the genus Helicostylum.

### 2. Preparation of Substrate

The fungus (strain JW-1) belonging to the genus Helicostylum obtained above in 1. was used. Using fungus that had been cultivated in advance and sterilized water, spore suspension (spore concentration: 8.5×10⁵ spores/mL) was prepared. Subsequently, an autoclaved rayon cloth was dipped in the prepared spore suspension and agitated gently. The dipped cloth was then transferred to a sterilized plastic container and dried overnight at 40° C. using a forced-air dryer to obtain a substrate.

### 3. Fish Meat Preservation

A 7.0 kg fillet (with skin attached) of tuna (farmed) was used as the fish meat. The substrate prepared above in 2. was wrapped around the surface of the tuna. Subsequently, a meat wrapper (made of rayon) was wrapped around the outside of the substrate. The tuna wrapped in the substrate and the meat wrapper was stored in a refrigerator (under a no airflow environment and under dehumidification conditions) at 4° C. for 25 days. In those cases where large numbers of drips exuded from the tuna, the meat wrapper was rewrapped periodically. After the storage period, when the substrate was removed from the fish meat, the vivid red color had been maintained, and a tendency for good suppression of discoloration was observed. There were also no cultivation odors, and the texture was moist. Further, the fish meat had a nutty flavor characteristic of an aged state.

### Example 2

Slices of tuna (farmed) that had been cut to about 0.5 kg and had the skin removed were wrapped with a substrate and a meat wrapper that had been prepared using the same method as that described in 2. of Example 1, the slices were then stored in a refrigerator (under a no airflow environment, 80 to 90% RH) at 4° C., and every 5 days for a period of 20 days, the substrate was removed and the slices were observed. The results revealed that for each slice, a vivid red color was maintained, and a tendency for good suppression of discoloration was shown. There were also no cultivation odors, and the texture was moist. Further, after 20 days, the slices had a nutty flavor characteristic of an aged state.

Based on the above results, it is clear that by wrapping the substrate provided with fungus around the surface of fish meat prior to storage, the fish meat can be stored under refrigeration while preventing spoiling, with particularly good prevention of discoloration of the fish meat and the generation of fish odors.

### Example 3

### 1. Pretreatment of Fish Meat

Salmon (from Norway, split longitudinally) was used as a sample. First, the two halves of the fish body were separated into individual pieces, the portions close to the head and tail were removed, and the remaining portions were used for testing. Each side portion was cut into four quarters, table salt equivalent to a weight of 5% was shaken over the belly side of each piece, and the pieces were left to stand for 10 minutes. The table salt was then washed off using tap water, and in a wet state, 70% ethanol was sprayed onto the belly side and left to stand for 5 minutes. The resulting pieces were used in the preservation test.

### 2. Fish Meat Preservation

The pieces corresponding with the same locations from each of the pretreated side portions were used as samples for day 0, day 7, day 14 and day 21, respectively. For one of the pieces, a substrate prepared using the same method as that described above in 2. of Example 1 was affixed to only the belly side of the piece, whereas for the other corresponding piece, a meat wrapper (a wrapper to which no fungal spores had been adhered: a control) that had been autoclaved (121° C., 15 minutes) was affixed in a similar manner. These samples were separated and stored in a business-use-constant-temperature constant-humidity refrigerator (4° C., humidity at least 80% but not more than 90%). The day 0 pieces were immediately vacuum-packed and stored in a freezer at −80° C. For each of the stored pieces, the substrate or meat wrapper was removed after the storage period, and the piece was vacuum-packed and stored at −80° C. in a similar manner.

### 3. Measurement of Produced Oxides

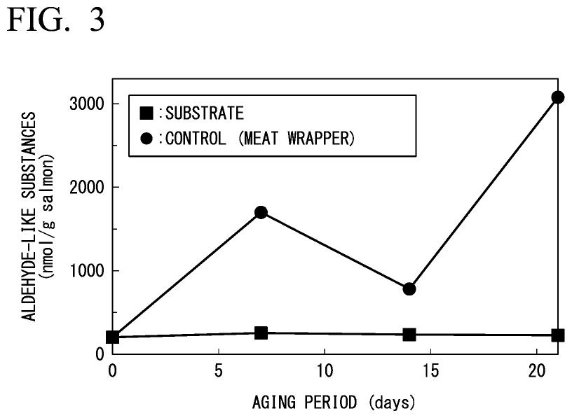

The degree of oxidation of the fish meat was evaluated by quantifying the amount of aldehyde-like substances produced by oxidation of fats using a TBARS (2-thiobarbituric acid reactive substance) assay. Each of the samples that had been frozen at −80° C. was allowed to thaw naturally at 4° C., and a 1 g sample from the belly side was used as the analysis sample. The 1 g sample and 9 mL of 1.15% KCl solution were placed in a test tube with a screw cap, and a homogenization was conducted in ice. To 0.5 mL of the resulting ground product-containing solution were added and mixed 0.3 mL of 1% phosphoric acid and 1.0 mL of a 0.67% thiobarbituric acid (TBA) solution, the resulting mixture was incubated for 45 minutes in a boiling bath to allow production of aldehyde-TBA complexes to proceed, and the mixture was then cooled in ice water. Subsequently, 4 mL of n-butanol was added to this sample and shaken vigorously for 10 minutes to extract the aldehyde-TBA complexes. The sample was then centrifuged at 3,000 rpm and room temperature for 10 minutes, thus separating the sample into a water phase and an n-butanol phase. The n-butanol phase was collected, and the absorbance at 535 nm and 520 nm of the TBA complexes contained therein was measured. Quantification of malondialdehyde (MAD) was determined by using a similar method to measure the absorbance of MAD produced in a 10 μM 1,1,3,3-tetraethoxypropane/methanol solution as a standard, and then calculating the amount of MAD using the formula below. In the following formula, f represents the difference in the absorbance at 535 nm and 520 nm (A₅₃₅-A₅₂₀) for the sample, and F represents the difference in the absorbance at 535 nm and 520 nm (A₅₃₅-A₅₂₀) for the standard. The results are shown in Table 2 and FIG. 3. The “substrate” refers to the sample to which was affixed the substrate having the adhered fungal spores, whereas the “control” refers to the sample to which was affixed the meat wrapper (having no adhered fungal spores).

Amount of aldehyde-like substances produced (nmol/g salmon) using MAD as a standard=f/F×10/0.5×9

TABLE 2

| Storage | Amount of Aldehyde-like | |
|---|---|---|
| period | Substances Produced (nmol/g salmon) | |
| (days) | Control | Substrate |
| 0 | 204 | 204 |
| 7 | 1,699 | 253 |
| 14 | 782 | 236 |
| 21 | 3,078 | 227 |

### 4. Results

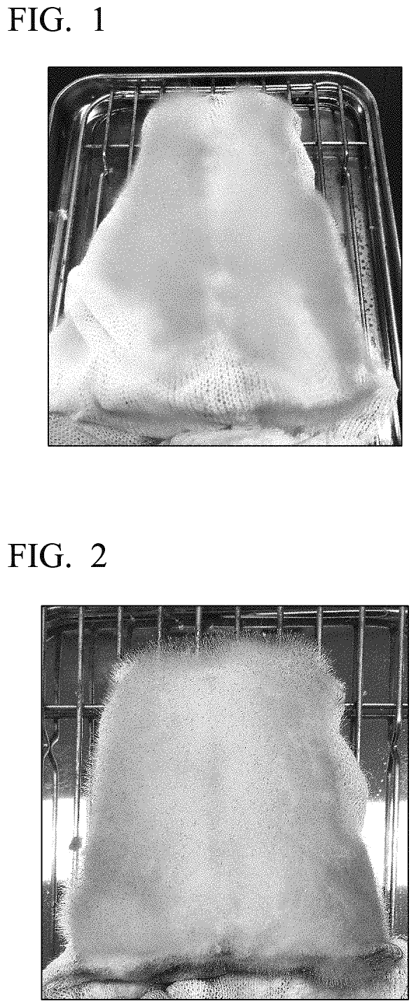

In the case of the samples to which the substrate having adhered fungal spores had been affixed, growth of mucor was confirmed on day 7, and the elongation of aerial hyphae was also confirmed (not shown in the drawings). By day 14, growth of the mucor had progressed to cover the entire piece of fish (see FIG. 1), and by day 21, the elongation of aerial hyphae of considerable length was confirmed (see FIG. 2). Furthermore, in terms of odor, no significant change in odor was noticed with the passage of time. On the other hand, in the control samples to which the meat wrapper had been affixed, an unpleasant odor thought to be caused by oxidation of fats had developed by day 7, and on day 21, the proliferation of mold-like microorganisms thought to be putrefying microorganism was observed.

Based on Table 2 and FIG. 3, it was evident that in the control samples to which the meat wrapper had been affixed, the amount of oxides indicated by the production of aldehyde-like substances increased significantly, whereas in the samples to which the substrate having adhered fungal spores had been affixed, even after 21 days storage, no increase in oxides was noticeable.

### INDUSTRIAL APPLICABILITY

By using the preservation method of an embodiment of the present invention, fish meat can be stored under refrigeration while preventing spoiling. By using the transportation method of an embodiment of the present invention, fish meat can be transported while preventing spoiling. By using the discoloration suppression method of an embodiment of the present invention, discoloration of the fish meat can be effectively suppressed. By using the fishy smell suppression method of an embodiment of the present invention, the occurrence of fish odors can be effectively suppressed. By using the oxidation suppression method of an embodiment of the present invention, oxidation of the fish meat can be effectively suppressed. Fish meat of an embodiment of the present invention exhibits little spoiling and displays good suppression of discoloration, fish odors and oxidation.

1. A fish meat oxidation suppression method comprising:
refrigerating fish meat at a temperature of at least 0° C. but not more than 6° C. in a state where the fish meat is in contact with a substrate,
wherein the substrate is provided with, at least in a portion that contacts the fish meat, fungi belonging to the genus Helicostylum, the genus Thamnidium, or the genus Mucor.

2. A fish meat preservation method comprising:
refrigerating fish meat at a temperature of at least 0° C. but not more than 6° C. in a state where the fish meat is in contact with a substrate,
wherein the substrate is provided with, at least in a portion that contacts the fish meat, fungi belonging to the genus Helicostylum, the genus Thamnidium, or the genus Mucor.

3. The preservation method according to claim 2,
wherein a material of the substrate is rayon or cotton.

4. A fish meat transportation method comprising:
refrigerating fish meat at a temperature of at least 0° C. but not more than 6° C. in a state where the fish meat is in contact with a substrate,
wherein the substrate is provided with, at least in a portion that contacts the fish meat, fungi belonging to the genus Helicostylum, the genus Thamnidium, or the genus Mucor.

5. A fish meat discoloration suppression method comprising:
refrigerating fish meat at a temperature of at least 0° C. but not more than 6° C. in a state where the fish meat is in contact with a substrate,
wherein the substrate is provided with, at least in a portion that contacts the fish meat, fungi belonging to the genus Helicostylum, the genus Thamnidium, or the genus Mucor.

6. A fish meat fishy smell suppression method A method of suppressing fishy smell of fish meat, comprising:
refrigerating fish meat at a temperature of at least 0° C. but not more than 6° C. in a state where the fish meat is in contact with a substrate,
wherein the substrate is provided with, at least in a portion that contacts the fish meat, fungi belonging to the genus Helicostylum, the genus Thamnidium, or the genus Mucor.

7. Fish meat having a surface covered with a substrate,
wherein the substrate is provided with, at least in a portion that contacts the fish meat, fungi belonging to the genus Helicostylum, the genus Thamnidium, or the genus Mucor.
